(19) United States
(12) Patent Application Publication
(10) Pub. No.: US 2020/0188015 A1
(43) Pub. Date: Jun. 18, 2020

(54) Introducer for Use with an RF Ablation Probe and Associated RF Ablation Probe Assembly

(71) Applicant: Avent, Inc., Alpharetta, GA (US)

(72) Inventor: Ruoya Wang, Decatur, GA (US)

(21) Appl. No.: 16/220,187

(22) Filed: Dec. 14, 2018

(51) Int. Cl. A61B 18/14

(52) U.S. Cl. CPC A61B 18/1492; A61B 2018/00577; A61B 18/1477

(57) ABSTRACT

A medical introducer for use in locating an energy delivery probe, such as an RF ablation probe, at a target location in tissue includes a proximal hub and an elongate cannula having a distal end and a proximal end, the proximal end connected to the hub. An elongate opening extends proximally from the distal end along a longitudinal axis of the cannula, the elongate opening defined by a long side of the cannula that extends to the distal end and an opposite open side. The elongate opening has a shape such that an active distal tip region of a probe inserted into the cannula is exposed longitudinally along the elongate opening and is covered by the long side along an opposite side of the distal tip region. A resulting lesion formed by use of the introducer with the probe is non-spherical and includes an enlarged lobe oriented along the elongate opening.

### FIELD OF THE INVENTION

The present invention relates generally to a medical cannula or introducer, and more particularly to an introducer used to guide a probe for applying energy for the treatment of tissue, for example in an RF ablation procedure

### BACKGROUND

Lower back injuries and chronic joint pain are major health problems resulting not only in debilitating conditions for the patient, but also in the consumption of a large proportion of funds allocated for health care, social assistance and disability programs. In the lower back, disc abnormalities and pain may result from trauma, repetitive use in the workplace, metabolic disorders, inherited proclivity, and/or aging. The existence of adjacent nerve structures and innervation of the disc are very important issues in respect to patient treatment for back pain. In joints, osteoarthritis is the most common form of arthritis pain and occurs when the protective cartilage on the ends of bones wears down over time.

A minimally invasive technique of delivering high-frequency electrical current has been shown to relieve localized pain in many patients. Generally, the high-frequency current used for such procedures is in the radiofrequency (RF) range, i.e. between 100 kHz and 1 GHz and more specifically between 300-600 kHz. The treatment of pain using high-frequency electrical current has been applied successfully to various regions of patients' bodies suspected of contributing to chronic pain sensations. In addition to creating lesions in neural structures, application of radiofrequency energy has also been used to treat tumors throughout the body.

The RF electrical current is typically delivered from a generator via connected electrodes that are placed in a patient's body, in a region of tissue that contains a neural structure suspected of transmitting pain signals to the brain. The electrodes generally include an insulated shaft with an exposed conductive tip to deliver the radiofrequency electrical current. Tissue resistance to the current causes heating of tissue adjacent resulting in the coagulation of cells (at a temperature of approximately 45° C. for small unmyelinated nerve structures) and the formation of a lesion that effectively denervates the neural structure in question. Denervation refers to affecting a neural structure's ability to transmit signals and usually results in the complete inability of a neural structure to transmit signals, thus removing the pain sensations.

To extend the size of a lesion, radiofrequency treatment may be applied in conjunction with a cooling mechanism, whereby a cooling means is used to reduce the temperature of the tissue near an energy delivery device, allowing a higher voltage to be applied without causing an unwanted increase in local tissue temperature. The application of a higher voltage allows regions of tissue further away from the energy delivery device to reach a temperature at which a lesion can form, thus increasing the size/volume of the lesion. In addition, radiofrequency ablation relies on the application of electrical energy to create heat tissue based on a closed-loop temperature feedback routine. More specifically, the ablation routine applies radiofrequency energy to reach and maintain preset temperature profiles. The temperature is typically measured through a thermocouple located at the distal tip of the active electrode. The output from the thermocouple must be filtered to reject the radiofrequency frequency prior to amplification.

Various procedures using RF probes for pain management or treatment use a cannula or “introducer” with a stylet to puncture the patient's skin and create a pathway to the target nerve location. Once the introducer is placed, the stylet is withdrawn from the introducer and the RF probe is inserted through the lumen in the introducer and secured to a proximal hub on the introducer, for example using a luer- lock fitting so that the active distal end of the probe extends beyond the distal end of the introducer.

Conventional introducers are typically made of a high strength metal, such as 304 grade stainless steel, and have a squared-off distal end. The introducer is used to establish the active tip length of the probe by creating an electrical barrier along the length of the probe shaft, wherein only the portion of the probe not covered by the introducer is electrically exposed and delivers RF energy into the tissue. For this reason, the metal introducer must be covered with an electrically insulating material, which is typically done by bonding a polymer polyamide material to the introducer blank. The polyamide creates a strong electrical barrier, but must be subsequently trimmed and bonded to the metal shaft with a relatively long cure time adhesive.

With the cooled ablation procedures discussed above, the RF probes are designed to create a relatively large, nearly spherical, axis-symmetric lesion that increases the probability of ablating the target nerve. This type of lesion also allows for an angle-independent approach to the treatment site, making the procedure easier to perform. This type of lesion and procedure are optimal for the spine, hip, sacral, and other locations where there is substantial tissue surrounding the ablation site. However, in more superficial sites such as the knee, a spherical (or nearly spherical) lesion may not be optimal as the lesion has a higher probability of burning the skin and creating thermal eschars.

Thus, a new and improved introducer and RF ablation probe system that is more suited for superficial treatment sites would be a welcome advancement in the art.

### SUMMARY OF THE INVENTION

Objects and advantages of the invention will be set forth in part in the following description, or may be obvious from the description, or may be learned through practice of the invention.

In one aspect, the present invention is directed to a medical introducer for use in locating an energy delivery probe at a target location in tissue. For example, the introducer may be used with an RF ablation probe in performance of an RF ablation procedure to manage or treat pain. The introducer includes a proximal hub, and an elongate cannula having a distal end and a proximal end, the proximal end connected to the proximal hub. An elongate opening extends proximally from the distal end along a longitudinal axis of the cannula, the elongate opening defined by a long side of the cannula that extends to the distal end and an opposite open side. The elongate opening includes a shape such that an active tip region of a probe inserted into the cannula is exposed longitudinally along the elongate opening and is covered by the long side along an opposite side of the active tip region. With this configuration, a lesion formed by use of the introducer with the active probe is non-spherical and includes an enlarged lobe oriented along the elongate opening. Thus, the introducer and active probe can be oriented such that, with a superficial target site such as within a knee or other location, the shaped lesion is directed towards the target site and not outwardly towards the patient's skin. Damage to the skin and thermal eschars are thus minimized.

In a particular embodiment, a proximal end wall defines a proximal end of the elongate opening, wherein the proximal end wall is squared perpendicular to the longitudinal axis of the cannula. The proximal end wall has a depth measured along a diameter of the cannula. In certain embodiments, this depth may extend between one-third to two-thirds of the diameter of the cannula.

The elongate opening may be further defined by side edges that extend from the proximal end wall to the distal end of the cannula, wherein the side edges include a straight section that extends longitudinally from the proximal end wall. The edges are “straight” in that they are aligned with the longitudinal axis of the cannula.

The straight sections may extend to the distal end of the cannula in one embodiment. In an alternate embodiment, the side edges include a distal tapered section that extends from the longitudinally extending straight section to the distal end of the cannula. This tapered section may be linear or curved.

In still another embodiment, the side edges that extend from the proximal end wall to the distal end of the cannula may have a continuously curved profile from the proximal end wall to the distal end of the cannula. For example, the curved profile may include a first curved section and a second oppositely curved section.

In various embodiments, the proximal hub may include one or both of a tactile or visual marker configured thereon that is aligned with the long side of the cannula. For example, the marker may be a ridge or bump on the surface of the hub that is easily felt by the clinician when manipulating the introducer and probe. In this manner, the clinician can readily orient the long side of cannula towards the patient's skin.

The present invention also encompasses an RF ablation probe system for use in locating a radio frequency (RF) probe at a target location in tissue, wherein the system includes the introducer discussed above, as well as an RF probe assembly comprising an elongate shaft insertable through the hub and into the cannula, the elongate shaft comprising an active distal tip region. As discussed above, with this system, a lesion formed by use of the introducer with the RF probe assembly is non-spherical and has an enlarged lobe oriented along the elongate opening.

The introducer in the RF ablation probe system may include any one or combination of the features discussed above.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 is a diagram of a system for applying radiofrequency (RF) electrical energy to target tissue in a patient's body;

FIG. 2 is a perspective view of one embodiment of an introducer and stylet in accordance with the present disclosure;

FIG. 3 is a perspective view with the stylet of FIG. 2 inserted into the introducer, and also illustrates a marker on the introducer hub aligned with the long side of the introducer cannula;

FIG. 4 is a perspective view of the distal end of the introducer cannula;

FIGS. 5a and 5b are perspective views of the distal end of the introducer cannula with the shaft of the RF probe inserted into the introducer;

FIG. 6 is a side view of an embodiment of the introducer with the shaft of the RF probe inserted into the introducer;

FIG. 7 is a side view of an alternate embodiment of the introducer with the shaft of the RF probe inserted into the introducer;

FIG. 8 is a side view of still another embodiment of the introducer with the shaft of the RF probe inserted into the introducer; and

FIG. 9 is a side view of a different embodiment of the introducer with the shaft of the RF probe inserted into the introducer.

### DETAILED DESCRIPTION OF THE INVENTION

Reference will now be made in detail to one or more embodiments of the invention, examples of the invention, examples of which are illustrated in the drawings. Each example and embodiment is provided by way of explanation of the invention, and is not meant as a limitation of the invention. For example, features illustrated or described as part of one embodiment may be used with another embodiment to yield still a further embodiment. It is intended that the invention include these and other modifications and variations as coming within the scope and spirit of the invention.

Before explaining various embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

For the purposes of this invention, a lesion refers to any effect achieved through the application of energy to a tissue in a patient's body, and the invention is not intended to be limited in this regard. Furthermore, for the purposes of this description, proximal generally indicates that portion of a device or system next to or nearer to a user (when the device is in use), while the term distal generally indicates a portion further away from the user (when the device is in use).

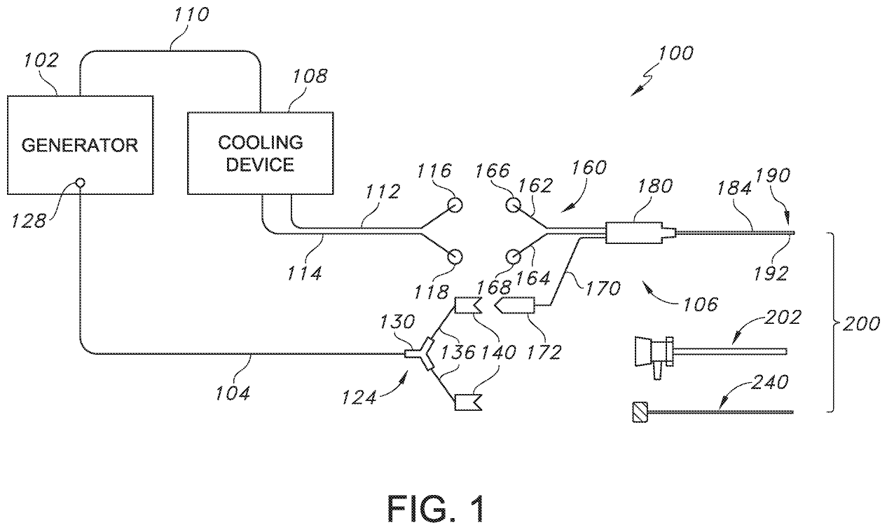

Referring now to the drawings, FIG. 1 illustrates a schematic diagram of a system 100 for application of energy, such as RF energy, to a target location within tissue of a patient, and is presented herein for purposes of describing an exemplary operating environment in which the present introducer and assembly may be used. The system 100 includes a generator 102, a cable 104, first and second probe assemblies 106 (only one probe assembly is shown), one or more cooling devices 108, a pump cable 110, one or more proximal cooling supply tubes 112, and one or more proximal cooling return tubes 114. The generator 102 may be a radiofrequency (RF) generator, or any other energy source, such as microwave energy, thermal energy, ultrasound, or optical energy. The generator 102 may include a display that displays various aspects of a treatment procedure, such as any parameters that are relevant to a treatment procedure, for example temperature, impedance, etc. and errors or warnings related to a treatment procedure. Alternatively, the generator 102 may include means of transmitting a signal to an external display. The generator 102 is operable to communicate with the first and second probe assemblies 106 and the one or more cooling devices 108. Such communication may be unidirectional or bidirectional depending on the devices used and the procedure performed.

In addition, as shown, a distal region 124 of the cable 104 may include a splitter 130 that divides the cable 104 into two distal ends 136 such that the probe assemblies 106 can be connected thereto. A proximal end 128 of the cable 104 is connected to the generator 102. This connection can be permanent, whereby, for example, the proximal end 128 of the cable 104 is embedded within the generator 102, or temporary, whereby, for example, the proximal end 128 of cable 104 is connected to generator 102 via an electrical connector. The two distal ends 136 of the cable 104 terminate in connectors 140 operable to couple to the probe assemblies 106 and establish an electrical connection between the probe assemblies 106 and the generator 102. In alternate embodiments, the system 100 may include a separate cable for each probe assembly 106 being used to couple the probe assemblies 106 to the generator 102.

The cooling device(s) 108 may include any means of reducing a temperature of material located at and proximate to one or more of the probe assemblies 106.

For example, the cooling devices 108 may include a pump assembly operable to circulate a fluid from the cooling devices 108 through one or more proximal cooling supply tubes 112, the probe assemblies 106, one or more proximal cooling return tubes 114, and back to the one or more cooling devices 108.

The system 100 may include a controller for facilitating communication between the cooling devices 108 and the generator 102 via a feedback control loop. The feedback control may be implemented, for example, in a control module which may be a component of the generator 102. In such embodiments, the generator 102 is operable to communicate bi-directionally with the probe assemblies 106 as well as with the cooling devices 108, wherein bi-directional communication refers to the capability of a device to both receive a signal from and send a signal to another device.

As an example, the generator 102 may receive temperature measurements from one or both of the first and second probe assemblies 106. Based on the temperature measurements, the generator 102 may perform some action, such as modulating the power that is sent to the probe assemblies 106. Thus, both probe assemblies 106 may be individually controlled based on their respective temperature measurements.

The pumps associated with the cooling devices 108 may communicate a fluid flow rate to the generator 102 and may receive communications from the generator 102 instructing the pumps to modulate this flow rate. With the cooling devices 108 turned off, any temperature sensing elements associated with the probe assemblies 106 would not be affected by the cooling fluid allowing a more precise determination of the surrounding tissue temperature to be made. In addition, when using more than one probe assembly 106, the average temperature or a maximum temperature in the temperature sensing elements associated with probe assemblies 106 may be used to modulate cooling.

The cooling devices 108 may reduce the rate of cooling or disengage depending on the distance between the probe assemblies 106. For example, when the distance is small enough such that a sufficient current density exists in the region to achieve a desired temperature, little or no cooling may be required. In such an embodiment, energy is preferentially concentrated between first and second energy delivery devices 192 through a region of tissue to be treated, thereby creating a strip lesion characterized by an oblong volume of heated tissue that is formed when an active electrode is in close proximity to a return electrode of similar dimensions.

The cooling devices 108 may also communicate with the generator 102 to alert the generator 102 to one or more possible errors and/or anomalies associated with the cooling devices 108. For example, if cooling flow is impeded or if a lid of one or more of the cooling devices 108 is opened. The generator 102 may then act on the error signal by at least one of alerting a user, aborting the procedure, and modifying an action.

Still referring to FIG. 1, the proximal cooling supply tubes 112 may include proximal supply tube connectors 116 at the distal ends of the one or more proximal cooling supply tubes 112. Additionally, the proximal cooling return tubes 114 may include proximal return tube connectors 118 at the distal ends of the one or more proximal cooling return tubes 114. In one embodiment, the proximal supply tube connectors 116 are female luer-lock type connectors and the proximal return tube connectors 118 are male luer-lock type connectors although other connector types are intended to be within the scope of the present invention.

In addition, as shown in FIG. 1, the probe assembly 106 may include a proximal region 160, a handle 180, a hollow elongate shaft 184, and an active distal tip region 190 that includes the one or more energy delivery devices 192. The elongate shaft 184 may be manufactured from an electrically conductive material and may be covered by an insulating material so that delivered energy remains concentrated at the energy delivery device 192 of the distal tip region 190. The proximal region 160 includes a distal cooling supply tube 162, a distal supply tube connector 166, a distal cooling return tube 164, a distal return tube connector 168, a probe assembly cable 170, and a probe cable connector 172. In such embodiments, the distal cooling supply tube 162 and distal cooling return tube 164 are flexible to allow for greater maneuverability of the probe assemblies 106, but alternate embodiments with rigid tubes are possible.

The distal supply tube connector 166 may be a male luer-lock type connector and the distal return tube connector 168 may be a female luer-lock type connector. Thus, the proximal supply tube connector 116 may be operable to interlock with the distal supply tube connector 166 and the proximal return tube connector 118 may be operable to interlock with the distal return tube connector 168.

The probe cable connector 172 may be located at a proximal end of the probe assembly cable 170 and may be operable to reversibly couple to one of the connectors 140, thus establishing an electrical connection between the generator 102 and the probe assembly 106. The probe assembly cable 170 includes one or more conductors to transmit RF current from the generator 102 to the one or more energy delivery devices 192, as well as to connect multiple temperature sensing devices to the generator 102 as discussed below.

The energy delivery devices 192 may include any means of delivering energy to a region of tissue adjacent to the distal tip region 190. For example, the energy delivery devices 192 may include an ultrasonic device, an electrode or any other energy delivery means and the invention is not limited in this regard. Similarly, energy delivered via the energy delivery devices 192 may take several forms including but not limited to thermal energy, ultrasonic energy, radiofrequency energy, microwave energy or any other form of energy. For example, in one embodiment, the energy delivery devices 192 may include an electrode. The active region of the electrode may be 2 to 20 millimeters (mm) in length and energy delivered by the electrode is electrical energy in the form of current in the RF range. The size of the active region of the electrode can be optimized for placement within an intervertebral disc; however, different sizes of active regions, all of which are within the scope of the present invention, may be used depending on the specific procedure being performed. In some embodiments, feedback from the generator 102 may automatically adjust the exposed area of the energy delivery device 192 in response to a given measurement such as impedance or temperature. For example, in one embodiment, the energy delivery devices 192 may maximize energy delivered to the tissue by implementing at least one additional feedback control, such as a rising impedance value.

FIG. 1 also depicts an introducer 202 and a stylet 240, wherein the combination of the RF probe assembly 106, the introducer 202, and the stylet 240 define an RF ablation probe system 200 in accordance with aspects of the present invention.

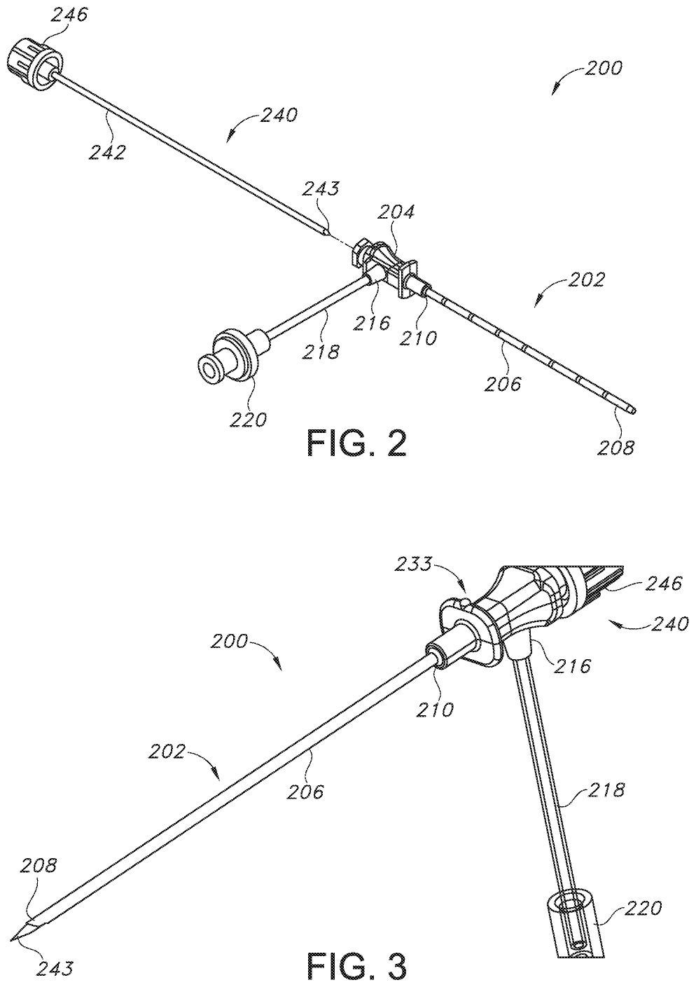

Referring to FIGS. 2 and 3, generally, the introducer 202 has a proximal end 210 configured with a hub 204 and a cannula 206 (defining an internal lumen) having a distal end 208. As understood in the art, the introducer 202 is operable to easily and securely couple with the RF probe assembly 106. For example, the proximal hub 204 is configured with a connector, such as a luer-lock connector, able to mate with the handle 180 of the RF probe assembly 106. The introducer cannula 206 is used to gain access to a tissue treatment site within a patient's body, wherein the elongate shaft 184 of the RF probe assembly 106 may be introduced to the treatment site through the longitudinal lumen of the introducer cannula 206.

The introducer cannula may be formed from metal and coated (e.g., dip coated) with a polymer layer such as PTFF (Polytetrafluoroethylene) or polyamide.

Function of the stylet 240 is understood in the art. Generally, the stylet 240 includes a proximal hub 246 fixed to an elongate needle 242 having a beveled tip 243 at the distal end thereof. The elongate needle 242 slides through the introducer 202 such that the stylet hub 246 connects to the introducer hub 204, for example via a luer-lock connection between the hubs 246 and 204, as depicted in FIG. 3. The tissue-piercing tip 243 at the distal end of the stylet needle 242 extends past the distal end 208 of the introducer cannula 206 to facilitate insertion of the introducer cannula 206 into the patient's body at the treatment target site. Various forms of stylets 240 are well known in the art and the present invention is not limited to include only one specific form. Further, the stylet 240 may be operable to connect to a power source and may therefore form part of an electrical current impedance monitor.

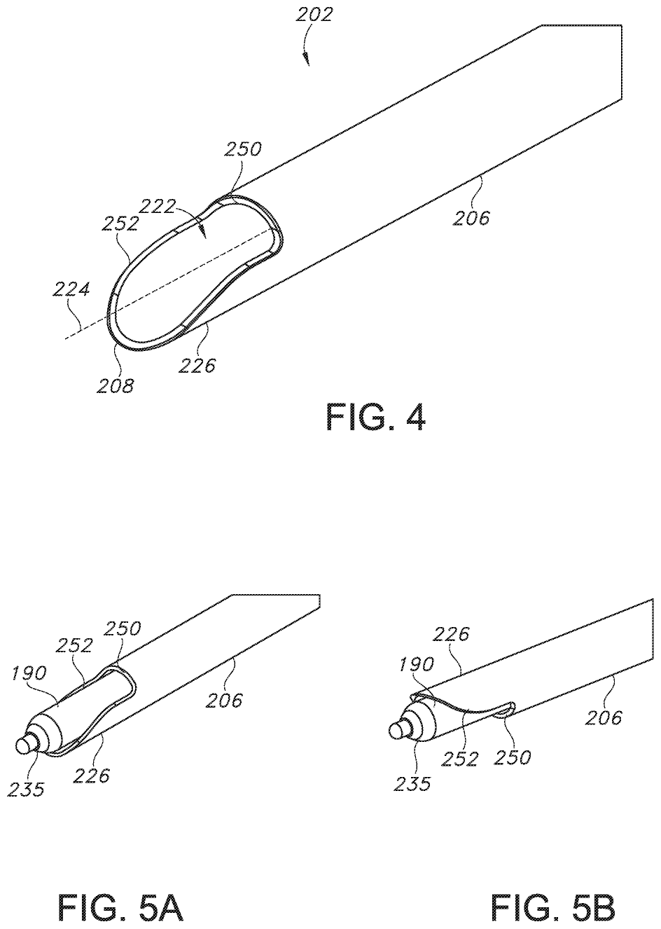

Referring to FIGS. 4-9, the cannula 206 is formed with an elongate opening 222 that extends proximally from the distal end 208 along a longitudinal axis 224 of the cannula 206. The elongate opening 222 is defined by a long side 226 of the cannula 206 that extends to the distal end 208 and an opposite longitudinally extending “open” side—meaning that the wall of the cannula 206 is not present at the open side. The absent section of the wall of the cannula 206 defines the elongate opening 222. Referring to FIGS. 5a and 5b, the elongate opening 222 has a shape or profile such that the active distal tip region 190 adjacent the distal end 235 of the of the shaft 184 (FIG. 1) of a probe assembly 106 inserted into the cannula 206 is exposed longitudinally along the elongate opening 222 and is covered by the long side 226 of the cannula 206 along an opposite side of the active tip region 190. With this unique configuration of the introducer cannula 206, a lesion formed by use of the introducer 202 with the active probe assembly 106 is non-spherical and includes an enlarged lobe oriented along the elongate opening 222. Thus, the introducer 202 and active probe assembly 106 can be oriented such that, with a superficial target site such as within a knee or other location, the shaped lesion is directed towards the target site and not outwardly towards the patient's skin. Damage to the skin and thermal eschars are thus minimized.

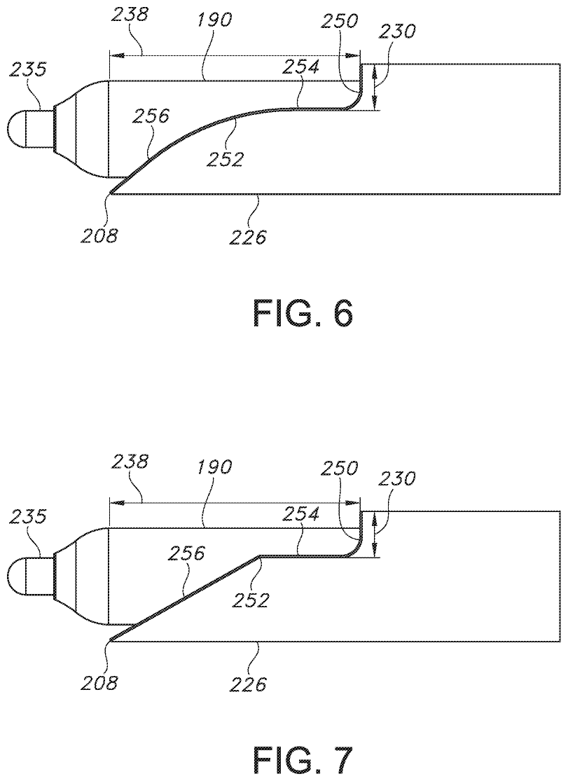

In particular embodiments depicted in FIGS. 4-9, a proximal end wall 250 defines a proximal end of the elongate opening 222. This proximal end wall 250 may be squared (as particularly seen in FIGS. 6 and 7) and perpendicular to the longitudinal axis 224 of the cannula 206. The proximal end wall 250 has a depth 230 measured along a diameter of the cannula, as particularly seen in FIGS. 6 and 7. In certain embodiments, this depth 230 may extend between one-third to two-thirds of the diameter of the cannula 206.

The elongate opening 222 may be further defined by side edges 252 that extend from the proximal end wall 250 to the distal end 208 of the cannula 206. These side edges 252 can have various shapes or profiles. For example, referring to FIGS. 6 and 7, the side edges 252 may include a straight section 254 that extends longitudinally from the proximal end wall 250 and are with the longitudinal axis 224 of the cannula 206.

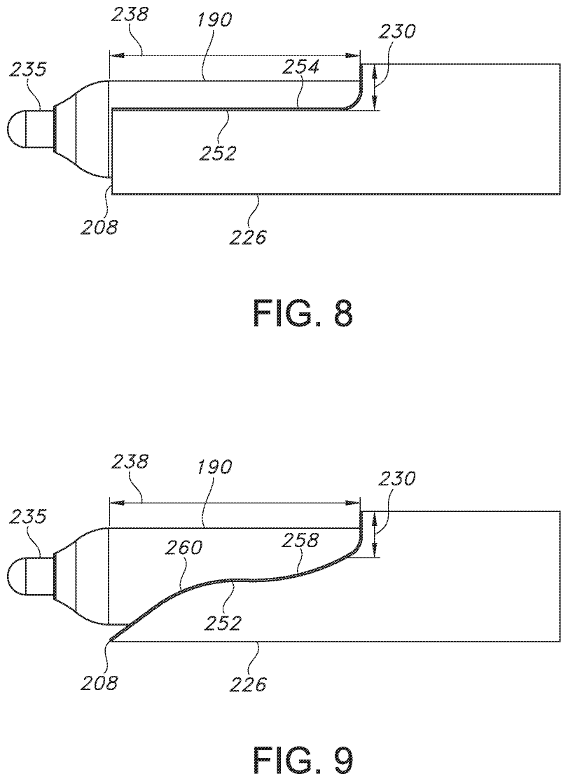

Referring to the embodiment of FIG. 8, the straight sections 254 may extend to the distal end 208 of the cannula 206.

In an alternate embodiment depicted in FIGS. 6 and 7, the side edges 254 include a distal tapered section 256 that extends from the longitudinally extending straight section 254 to the distal end 208 of the cannula 208. This distal tapered section 254 may be linear (FIG. 7) or curved (FIG. 6).

FIG. 9 depicts and embodiment wherein the side edges 252 that extend from the proximal end wall 250 to the distal end 208 of the cannula 206 may have a continuously curved profile. For example, the curved profile may include a first curved section 258 that merges with a second oppositely curved section 260.

Referring to FIG. 3, in various embodiments, the proximal hub 246 of the introducer may include one or both of a tactile or visual marker 233 configured thereon that is aligned with the long side of the cannula 206. For example, the marker 233 may be a ridge or bump on the surface of the hub that is easily felt by the clinician when manipulating the introducer 202 and probe 106 assembly. The marker 233 may be a contrasting stripe or other visual mark. In this manner, the clinician can readily orient the long side 226 of cannula 206 towards the patient's skin.

As discussed, the present invention encompasses an RF ablation probe system 200 (FIG. 1) for use in locating a radio frequency (RF) probe assembly 106 at a target location in tissue to treat or manage pain in a patient. The system 200 includes the introducer 202 discussed above, as well as an RF probe assembly 106 comprising an elongate shaft 184 insertable through the hub 204 and into the cannula 206 of the introducer 202, the elongate shaft comprising an active distal tip region 190. As discussed above, with this system, a lesion formed by use of the introducer 202 with the RF probe assembly 106 is non-spherical and has an enlarged lobe oriented along the elongate opening. The characteristics and features of the introducer 202 discussed above are applicable to the introducer 202 included with the RF ablation probe system 200.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A medical introducer for use in locating an energy delivery probe at a target location in tissue, comprising:
a proximal hub;
an elongate cannula having a distal end and a proximal end, the proximal end connected to the hub;
an elongate opening extending proximally from the distal end along a longitudinal axis of the cannula, the elongate opening defined by a long side of the cannula that extends to the distal end and an opposite open side, the elongate opening comprising a shape such that an active distal tip region of a probe inserted into the cannula is exposed longitudinally along the elongate opening and is covered by the long side along an opposite side of the distal tip region; and
wherein a resulting lesion formed by use of the introducer with the probe is non-spherical and includes an enlarged lobe oriented along the elongate opening.

2. The medical introducer of claim 1, comprising a proximal end wall that defines a proximal end of the elongate opening, the proximal end wall squared perpendicular to the longitudinal axis of the cannula.

3. The medical introducer of claim 2, wherein the proximal end wall has a depth that extends between one-third to two-thirds of a diameter of the cannula.

4. The medical introducer of claim 2, comprising side edges extending from the proximal end wall to the distal end of the cannula, the side edges comprising a longitudinally extending straight section adjacent the proximal end wall.

5. The medical introducer of claim 4, wherein the longitudinally extending straight section extends to the distal end of the cannula.

6. The medical introducer of claim 4, wherein the side edges comprise a distal tapered section that extends from the longitudinally extending straight section to the distal end of the cannula.

7. The medical introducer of claim 2, comprising side edges extending from the proximal end wall to the distal end of the cannula, the side edges comprising a continuously curved profile from the proximal end wall to the distal end of the cannula.

8. The medical introducer of claim 7, wherein the curved profile comprises a first curved section and a second oppositely curved section.

9. The medical introducer of claim 1, wherein the proximal hub comprises one or both of a tactile or visual marker configured thereon that is aligned with the long side of the cannula.

10. An RF ablation probe system for use in locating a radio frequency (RF) probe at a target location in tissue, comprising:
an introducer, the introducer comprising
a proximal hub;
an elongate cannula having a distal end and a proximal end, the proximal end connected to the hub;
an RF probe assembly comprising an elongate shaft insertable through the hub and into the cannula, the elongate shaft comprising an active distal tip region;
the elongate cannula further comprising an elongate opening extending proximally from the distal end along a longitudinal axis of the cannula, the elongate opening defined by a long side of the cannula that extends to the distal end and an opposite open side, the elongate opening comprising a shape such that the active distal tip region of the elongate shaft is exposed longitudinally along the elongate opening and is covered by the long side along an opposite side of the active distal tip region; and
wherein a resulting lesion formed by use of the introducer with the RF probe assembly is non-spherical and includes an enlarged lobe oriented along the elongate opening.

11. The RF ablation probe system of claim 10, wherein the introducer comprises a proximal end wall that defines a proximal end of the elongate opening, the proximal end wall squared perpendicular to the longitudinal axis of the cannula.

12. The RF ablation probe system of claim 11, wherein the proximal end wall has a depth that extends between one-third to two-thirds of a diameter of the cannula.

13. The RF ablation probe system of claim 11, comprising side edges extending from the proximal end wall to the distal end of the cannula, the side edges comprising a longitudinally extending straight section adjacent the proximal end wall.

14. The RF ablation probe system of claim 13, wherein the longitudinally extending straight section extends to the distal end of the cannula.

15. The RF ablation probe system of claim 13, wherein the side edges comprise a distal tapered section that extends from the longitudinally extending straight section to the distal end of the cannula.

16. The RF ablation probe system of claim 11, comprising side edges extending from the proximal end wall to the distal end of the cannula, the side edges comprising a continuously curved profile from the proximal end wall to the distal end of the cannula.

17. The RF ablation probe system of claim 16 wherein the curved profile comprises a first curved section and a second oppositely curved section.

18. The RF ablation probe system of claim 10, wherein the proximal hub comprises one or both of a tactile or visual marker configured thereon that is aligned with the long side of the cannula.
